(19) United States
(12) Patent Application Publication
(10) Pub. No.: US 2012/0265301 A1
(43) Pub. Date: Oct. 18, 2012

(54) INTRAOSSEOUS FIXATION ASSEMBLY FOR AN OSTEOTOMY AND METHOD OF USE

(76) Inventors: Matt Demers, Hoboken, NJ (US); Jeff Tyber, Bethlehem, PA (US); Brian Donley, Solon, OH (US); Jamy Gannoe, West Milford, NJ (US)

(21) Appl. No.: 13/088,333

(22) Filed: Apr. 16, 2011

(51) Int. Cl. A61F 2/28; A61B 17/86

(52) U.S. Cl. 623/16.11; 606/301

(57) ABSTRACT

An intraosseous fixation assembly for insertion into an osteotomy includes a wedge member for creating a resultant angle in the osteotomy, where the wedge member includes a body portion and a plurality of apertures. The fixation assembly also includes a first member adapted for coupling to the body portion at a first angle and a second member adapted for coupling to the body portion at a second angle, where each of the first and the second angles is at a divergent angle with respect to the body portion. In addition, the plurality of apertures includes at least a first aperture and a second aperture, where the first aperture is adapted for receiving the first member, and the second aperture is adapted for receiving the second member.

### FIELD OF THE INVENTION

This invention relates to the field of orthopedic implant devices, and more particularly, to an intraosseous fixation assembly that is used for angular correction of bones.

### BACKGROUND OF THE INVENTION

Orthopedic implant devices, such as intramedullary nails, plates, rods and screws are often used to repair or reconstruct bones and joints affected by trauma, degeneration, deformity and disease, such as Hallux Valgus deformities, failed Keller Bunionectomies, Rheumatoid Arthritis, lapidus bunionectiomies, proximal calcaneal bunionectomies, and other similar types of indications. In any surgical procedure, infections and wound complications are a major concern, and even more so in the aforementioned procedures. Wound closure is technically demanding for the surgeon, and devices, such as plates or exposed screws that add surface prominence, add to the difficulty by requiring greater tissue tension during incision reapproximation. This increases the risk of postoperative wound infections and dehiscence that may ultimately result in limb amputation.

Various implants have been utilized for surgical treatment of these affected bones. As an example, implants have been utilized to treat hallux valgus deformities in the foot bones through bone screws and plates. These bone screws and plate implants are commonly used in procedures to fuse several bones in the foot, for example, the first metatarsal bone and the first phalangeal bone in hallux valgus deformities (bunionectomy), failed keller bunionectomies, rheumatoid arthritis, and other types of indications. In a hallux valgus deformity correction, screws and plates may be used to straighten the phalanx relative to the first metatarsal and adjacent phalanges, reposition the sesamoid bones beneath the first metatarsal bone, or correct any abnormal bowing or misalignment within the first phalanx bones. While these devices allow fixation and promote fusion, they are neither effective in realigning bones, particularly in bunionectomy procedures, nor do they deliver uniform compression at various predetermined angles of compression.

Particularly, screw implants are ineffective in delivering sufficient angular correction for hallux valgus deformities to the bones in the foot, while preventing screw head break out, or delivering effective bending resistance. Moreover, hard to control dorsiflexion and valgus angles as well skin irritation from proximity to the skin prevents these screw implants from being readily utilized for surgical treatment. Yet further, plate implants used with bone screws too have the same drawbacks. The fixed varus and valgus angles limit effectiveness of these plate implants. Further, the lack of direct compression across the bones of the joint and skin irritations as a result of the proximity of the screw head to the skin also limit the effectiveness of these implants. Moreover, the presence of arthritis in the hallux joint limits the plates implants from being an effective remedy.

There is therefore a need for an intraosseous fixation assembly for angular correction that resides substantially within a bone and which overcomes some or all of the previously delineated drawbacks of prior orthopedic implant devices.

### SUMMARY OF THE INVENTION

An object of the invention is to overcome the drawbacks of previous inventions.

Another object of the invention is to provide a novel and useful fixation assembly that may be utilized to treat the bones in a human foot.

Another object of the invention is to provide a system for treating bunionectomies using an intraosseous fixation assembly.

Another object of the invention is to provide a wedge member for lapidus, calcaneal slide, or metatarsal osteotomies.

In a first non-limiting aspect of the invention, an intraosseous fixation assembly includes a wedge member having a body portion and a plurality of apertures. Also, the fixation assembly includes a first member adapted for coupling to the body portion at a first fixed angle and a second member adapted for coupling to the body portion at a second fixed angle.

In a second non-limiting aspect of the invention, a method for angular correction of a bone includes several steps. In one step, a wedge member having a first aperture and a second aperture is provided. Another step includes forming an osteotomy in a first metatarsal bone and spreading the osteotomy to create a cavity. In another step, a targeting guide assembly is coupled to the wedge member. Another step includes inserting the wedge member into the cavity at a predetermined depth. Another step includes inserting a drill member into the targeting guide assembly and into the first aperture. Another step includes forming a first hole in the metatarsal bone. Another step includes inserting a first lag screw member into the first aperture and into the first hole and compressing the metatarsal bone. Another step includes inserting the drill member into the targeting guide assembly and into the second aperture. In another step, a second hole is formed in the metatarsal bone. Another step includes inserting a second lag screw member into the second aperture and into the second hole and compressing the metatarsal bone.

In a third non-limiting aspect of the invention, an intraosseous fixation system for angular correction of a metatarsal bone includes a wedge member having a body portion and a plurality of apertures, a first screw member adapted for coupling to the body portion at a first fixed angle, a second screw member adapted for coupling to the body portion at a second fixed angle, and a targeting guide assembly adapted for coupling to the wedge member.

### BRIEF DESCRIPTION OF THE DRAWINGS

A further understanding of the invention can be obtained by reference to a preferred embodiment set forth in the illustrations of the accompanying drawings. Although the illustrated embodiment is merely exemplary of systems and methods for carrying out the invention, both the organization and method of operation of the invention, in general, together with further objectives and advantages thereof, may be more easily understood by reference to the drawings and the following description. The drawings are not intended to limit the scope of this invention, which is set forth with particularity in the claims as appended or as subsequently amended, but merely to clarify and exemplify the invention.

For a more complete understanding of the invention, reference is now made to the following drawings in which:

FIG. 1 is a perspective view of a fixation assembly inserted into a foot according to the preferred embodiment of the invention;

FIG. 2A is a perspective view of a fixation assembly shown in FIG. 1 according to the preferred embodiment of the invention;

FIG. 3A is a perspective view of a wedge member shown in FIG. 2A according to the preferred embodiment of the invention;

FIG. 3B is a side view of the wedge member shown in FIG. 2A according to the preferred embodiment of the invention;

FIG. 3C is a front view of the wedge member shown in FIG. 2A according to the preferred embodiment of the invention;

FIG. 4A is a perspective view of a foot with an osteotomy provided in a surgical method according to the preferred embodiment of the invention;

FIG. 4B is a perspective view of a foot with a trial inserter provided in a surgical method according to the preferred embodiment of the invention;

FIG. 4C is a perspective view of a foot with a wedge member coupled to an instrument provided in a surgical method according to the preferred embodiment of the invention;

FIG. 4D is a perspective view of a wedge member coupled to an instrument according to the preferred embodiment of the invention;

FIG. 4E is a perspective view of a foot with a drill coupled to the instrument of FIGS. 4C-4D according to the preferred embodiment of the invention;

FIG. 4F is a perspective view of a drill coupled to the instrument of FIGS. 4C-4D according to the preferred embodiment of the invention;

FIG. 4G is a perspective view of a lag screw member inserted into the foot according to a surgical method according to the preferred embodiment of the invention;

FIG. 4H is a perspective view of a drill coupled to the instrument of FIGS. 4C-4D according to the preferred embodiment of the invention;

FIG. 5 is a flow chart illustrating the surgical method of coupling the fixation assembly shown in FIGS. 1-4E to a metatarsal bone in a patient's foot according to the preferred embodiment of the invention;

FIG. 6 is a perspective view of a fixation assembly according to an alternate embodiment of the invention;

FIG. 7 is a perspective view of a fixation assembly with tapered and polyaxial screw members according to an alternate embodiment of the invention;

FIG. 8 is a perspective view of a fixation assembly with Kirschner wires according to an alternate embodiment of the invention.

FIG. 9 is a perspective view of a fixation assembly with lag screw member according to an alternate embodiment of the invention; and

FIG. 10 is a perspective view of a fixation assembly with a lag screw member and a holding spike according to an alternate embodiment of the invention.

### DETAILED DESCRIPTION OF THE INVENTION

The invention may be understood more readily by reference to the following detailed descriptions of preferred embodiment of the invention. However, techniques, systems, and operating structures in accordance with the invention may be embodied in a wide variety of forms and modes, some of which may be quite different from those in the disclosed embodiment. Consequently, the specific structural and functional details disclosed herein are merely representative, yet in that regard, they are deemed to afford the best embodiment for purposes of disclosure and to provide a basis for the claims herein, which define the scope of the invention. It must be noted that, as used in the specification and the appended claims, the singular forms “a”, “an”, and “the” include plural referents unless the context clearly indicates otherwise.

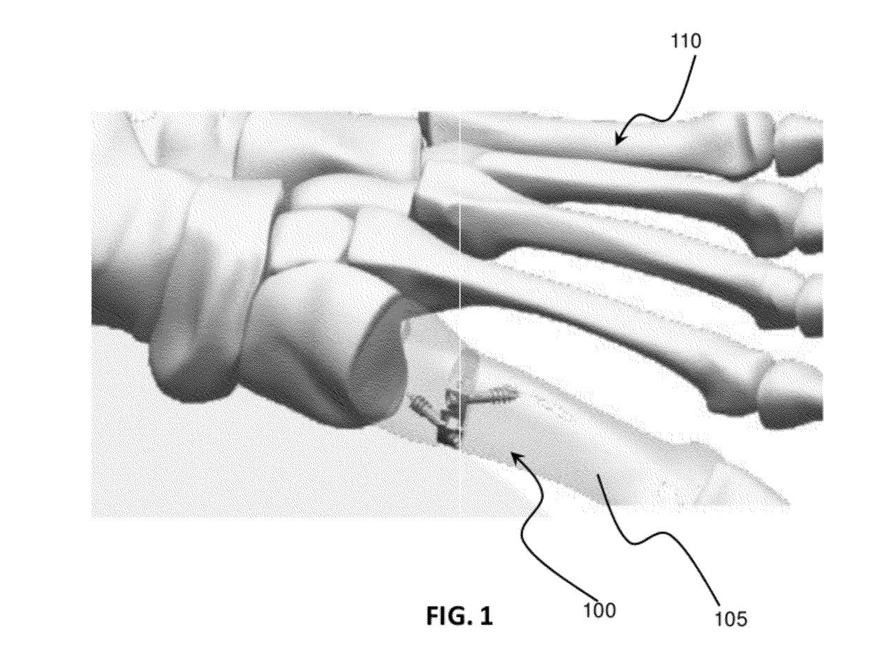

Referring now to FIG. 1, there is shown an intraosseous fixation assembly 100, which is made in accordance with the teachings of the preferred embodiment of the invention. As shown, the fixation assembly 100 is adapted to be inserted into an osteotomy that may be formed, in one example, laterally in the first metatarsal bone 105 and is subsequently coupled to the metatarsal bone 105 in order for providing angular intraosseous correction of the proximal metatarsal bunionectomy of the foot 110. In other non-limiting embodiments, the fixation assembly 100 may be inserted intraosseously into other metatarsal bones, the cuneiform, the calcaneus, the cuboid, or other ankle bones so as to perform a calcaneal slide, lapidus, or distal metatarsal osteotomy. It should be appreciated that the fixation assembly 100 may be provided at various lengths in order to be utilized or the internal fixation of a variety of bone sizes in the human body. The fixation assembly 100 may be made from a titanium material although, in other non-limiting embodiments, the fixation assembly 100 may be made from stainless steel (SST), Polyether ether ketone (PEEK), Nickel titanium (NiTi), Cobalt chrome, or other similar types of materials.

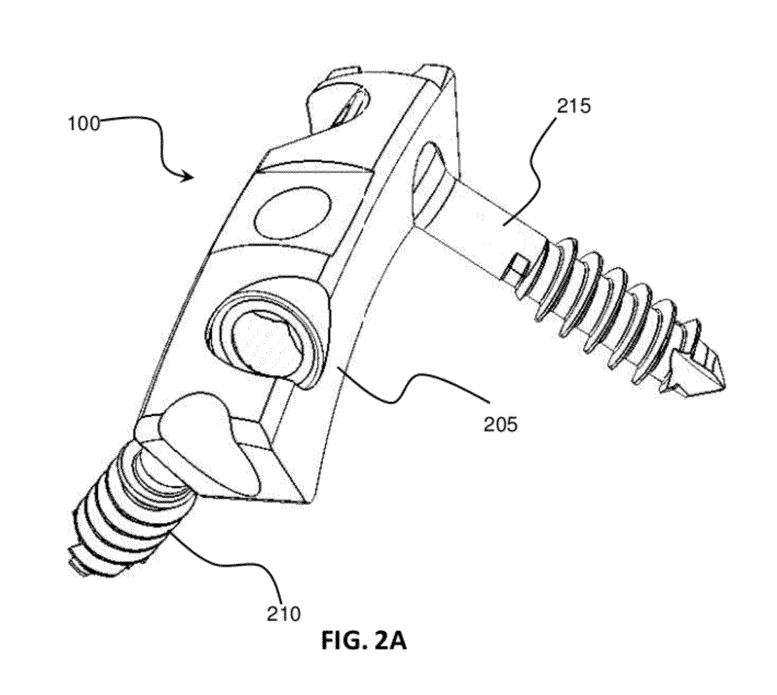

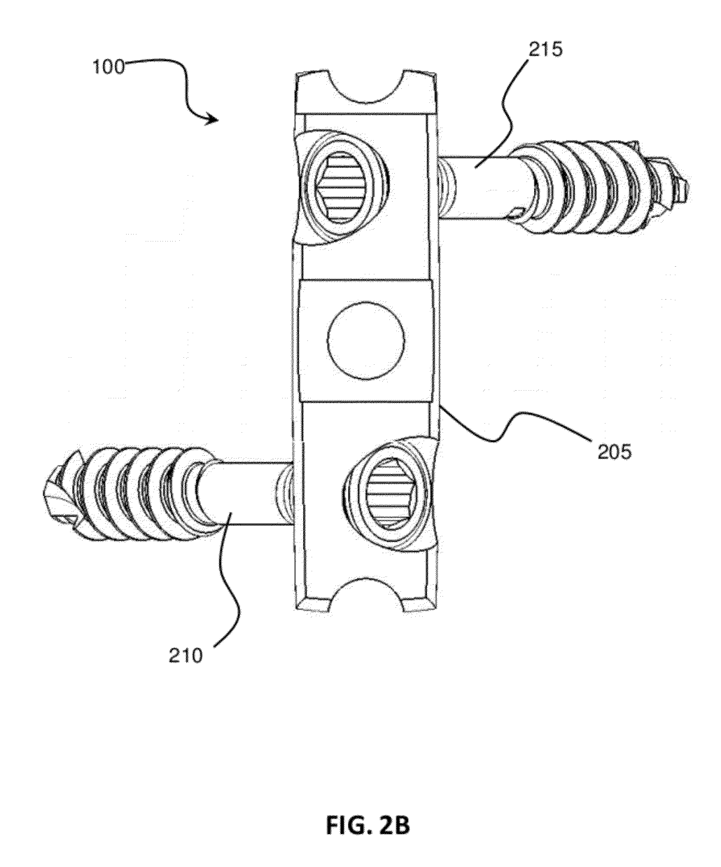

Referring next to FIGS. 2A-2B, the fixation assembly 100 includes a “wedge-shaped” member 205 (hereinafter “wedge member 205”) coupled to a plurality of oblique cortical lag screw members 210 and 215. Each of the lag screws 210, 215 includes an elongated body having a threaded screw portion at one end and a tapered head portion at an opposed second end. The tapered head portion includes a morse taper at the second end to create an interference lock with member 205, although in another embodiment, the lag screw members 210, 215 may be provided with a non-locking polyaxial head at the second end. The lag screw members 210, 215 may be cannulated or have a solid body and may be made from a Titanium material, although, in other non-limiting embodiments, these lag screw members 210, 215 may be made from SST, PEEK, NiTi, Cobalt chrome or other similar types of materials. The lag screw members 210, 215 are preferably locking screws and are provided to be coupled to the wedge member 205 at predetermined fixed angles with respect to wedge member 205. In other non-limiting examples, any or both of the lag screws 210, 215 may be non-locking screws for coupling the wedge member 205 to underlying bone.

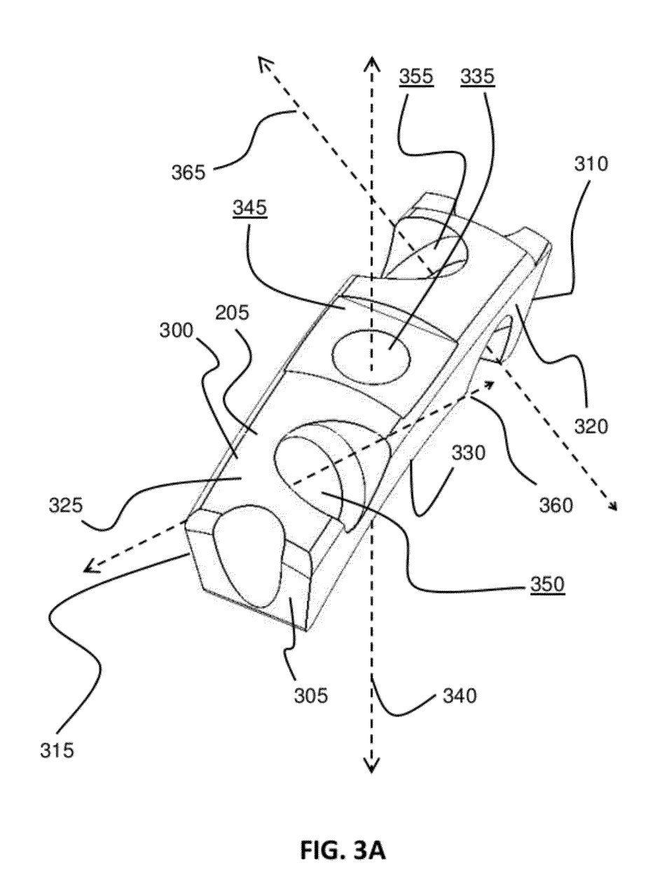

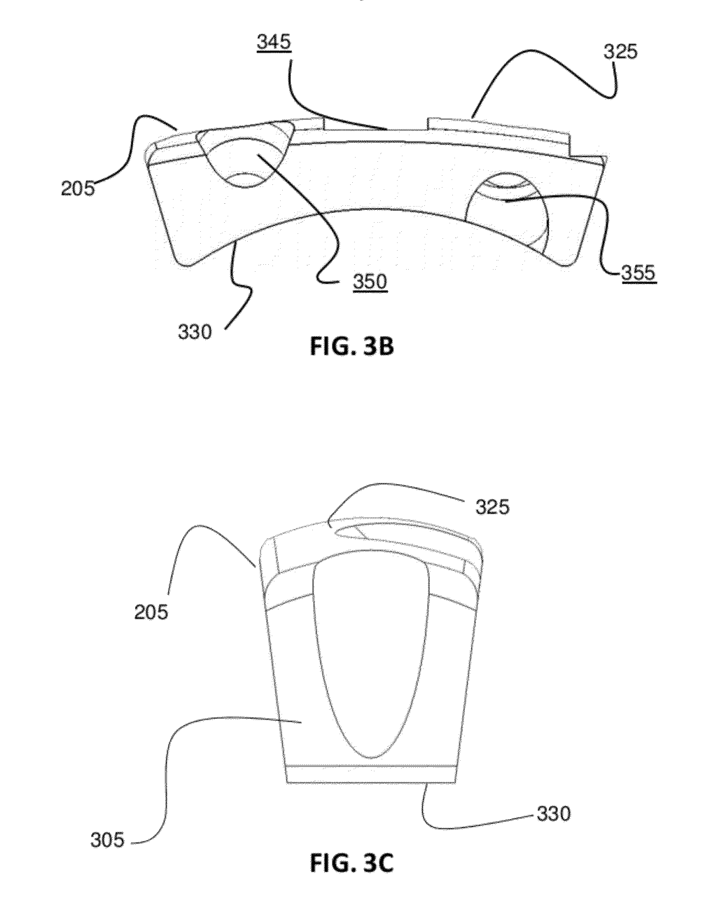

Further, lag screw members 210, 215 are inserted into apertures, which are provided at divergent and predetermined angles (shown in FIGS. 3A-3C), and thereafter into bone in order to provide for cortical purchase of the screw members 210, 215 into underlying bone at these divergent angles. In the example shown, the fixation assembly 100 is coupled to the metatarsal bone 105 in the human foot 110 (shown in FIG. 1). The divergent angles may be formed at predetermined fixed angles in the wedge member 205 depending on the bone segments into which the fixation assembly 100 is required to be inserted. These angles may be predetermined by, for example, a surgeon to fix the bones in the foot 110. In one non-limiting embodiment, the angle may be about 0 degrees to about 90 degrees. As such, each wedge member 205 is provided with a particular fixed angle for correcting specific bone or bones. The divergent angles between the wedge member 205 and the lag screw members 210, 215 provides the fixation assembly 100 with a means to apply compression at multiple points within the metatarsal bone 105, thereby increasing the stability of the fixation assembly 100, and preventing the rotation of the wedge member 205 within the metatarsal bone 105 (FIG. 1). It should be appreciated that the wedge member 205 may provided in various sizes so as to accommodate bones of various sizes and shapes. It should also be appreciated that the lag screw members 210, 215 may be provided at varying lengths for the internal fixation of a variety of bone sizes in the human body. The lag screw members 210, 215 may be coated with an osteoconductive material, such as, for example, plasma spray or other similar types of porous materials that is capable of supporting or encouraging bone ingrowth into this porous material.

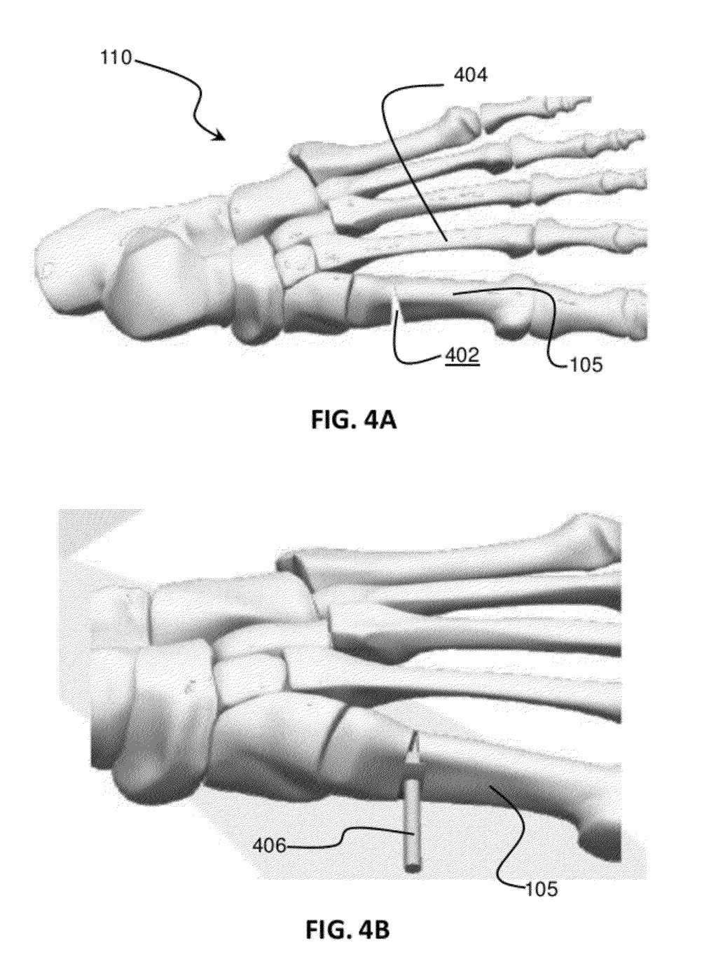

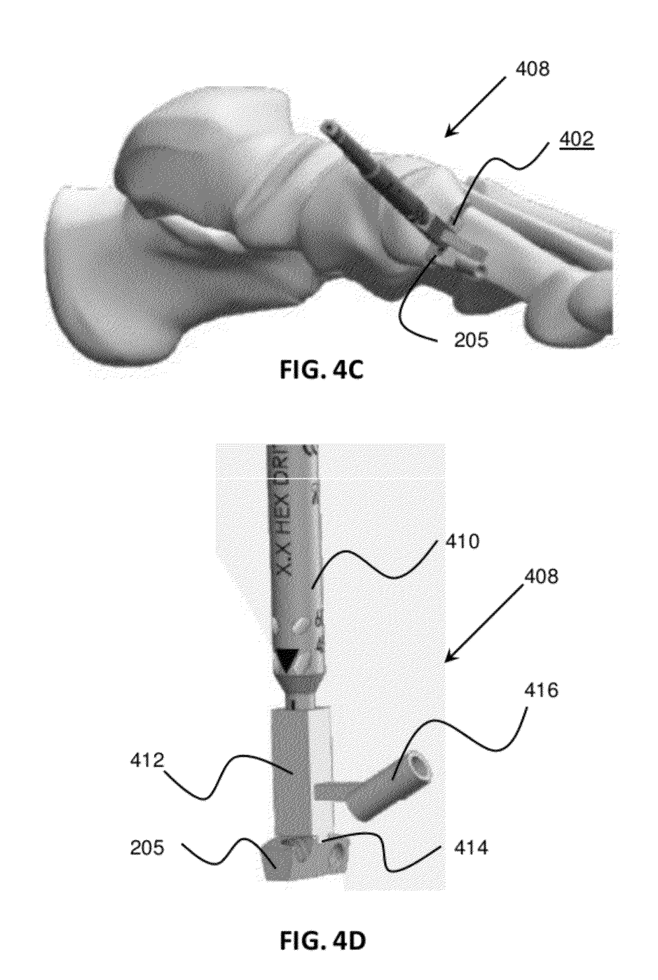

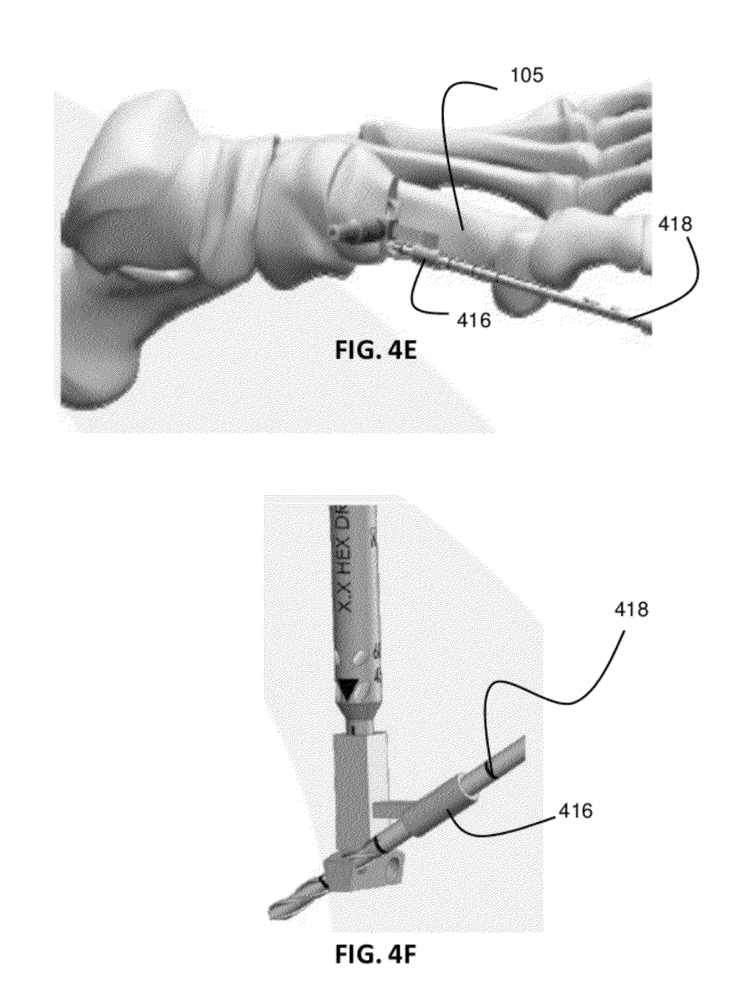

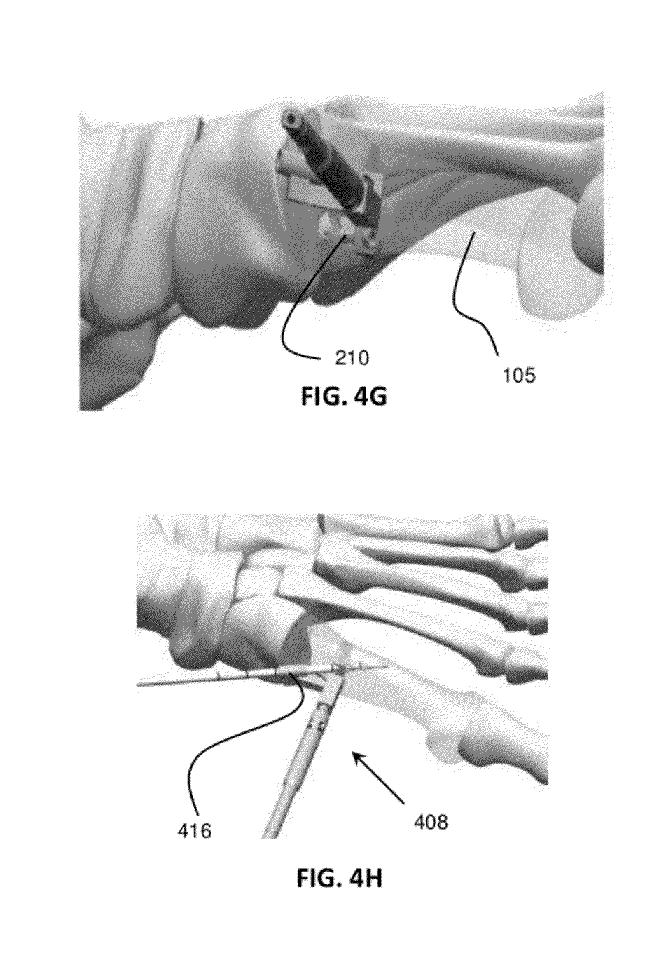

As shown in FIGS. 3A-3C, wedge member 205 has a generally “wedge” shape and includes a curved cross-section for intraosseous fixation within bone. Particularly, as shown in FIG. 3A, wedge member 205 has a solid and generally curved body portion 300 with a length that extends from tapered side 305 to tapered side 310, and a width that extends from tapered side 315 to tapered side 320, thereby giving body portion 300 substantially a wedge shape (i.e., body portion 300 has a perimeter that decreases from top surface 325 (FIG. 3A-3C) to opposed bottom surface 330 (FIG. 3A-3C). As shown in FIG. 3B, top surface 325 has a first radius of curvature that is larger than the second radius of curvature of bottom surface 330. However, in another non-limiting embodiment, top surface 325 and bottom surface 325 may both be substantially flat. Referring back to FIG. 3A, wedge member 205 includes a through-hole or aperture 335 that is aligned along the vertical axis 340. As shown, aperture 335 traverses through substantially the center of body portion 300, and extends orthogonally from top surface 325 and further emanates from the opposed bottom surface 330. Body portion 300 also includes a groove or slot 345 (FIGS. 3A-3B) surrounding hole 335 and formed in a plane that is orthogonal to the axis 340, and traversing the top surface 325 from tapered side 315 to tapered side 320. The slot 345 and aperture 335 cooperatively receive an instrument 408 (for example, shown in FIG. 4D), to actively hold the wedge member 205 within the osteotomy for insertion of at least one of the lag screw members 210, 215 (FIGS. 2A-2B) into underlying bone, which is shown and described below. In another non-limiting embodiment, the slot 345 may not be provided, and the instrument 408 utilizes the aperture 335 to hold the wedge member 205 against in the osteotomy.

Also shown in FIGS. 3A-3B, body portion 300 includes a plurality of apertures 350 and 355, which are aligned along axes 360 and 365 respectively (i.e., aperture 350 is aligned along axis 360 and aperture 355 is aligned along axis 365). Particularly, aperture 350, being aligned with axis 360, causes aperture 350 to be at a fixed and divergent angle with respect to the plane parallel to the surface of slot 345 (FIG. 3A) while aperture 355, being aligned with axis 365, causes aperture to be at a fixed and divergent angle with respect to the plane parallel to the surface of slot 345 (FIG. 3A). Also, as shown in FIG. 3A, apertures 350 and 355, being aligned on these axes 360 and 365 respectively, causes aperture 350 to originate from top surface 325 of body portion 300 and emanate substantially from tapered side 315, while also causing aperture 355 to originate from top surface 325 and emanate substantially from tapered side 320. The axes 360, 365 for the respective apertures 350, 355 may be predetermined during manufacture based on the surgical needs of a surgeon and the size of bones into which the wedge member 205 is being inserted. The apertures 350 and 355 are provided to receive tapered locking screws 210 and 215 respectively (FIGS. 2A-2B). Specifically, apertures 350, 355 receive locking screws 210 and 215 causing a locked interference fit between wedge member 205 and the tapered head of lag screw members 210, 215 as lag screw members 210, 215 (FIGS. 2A-2B) are inserted into apertures 350, 355, and lag screw members 210, 215 are further rotated causing these screw members 210, 215 to purchase into bone. It should be appreciated that wedge member 205 is provided to be a bone substitute material for angular correction of bones and may substantially fill the interstitital void volume created by an osteotomy. It should also be appreciated that wedge member 205 may be coated with a porous undercoating such as plasmacoated Titanium, mesh Titanium, or other similar types of osteoconductive materials, in order to provide an osteoconductive surface for fixating wedge member 205 to bone. In other non-limiting embodiments, wedge member 205 may be infused with hydroxylapatite or other similar types of growth hormones to increase connection to the bone.

As shown in FIGS. 1 and 4A-5, the fixation assembly 100 may be utilized by a surgeon to treat a metatarsal bunionectomy in the foot by inserting the fixation assembly 100 into an interstitial wedge made by an osteotomy. In other embodiments, the fixation assembly 100 may also be utilized on substantially any other bone in the human body.

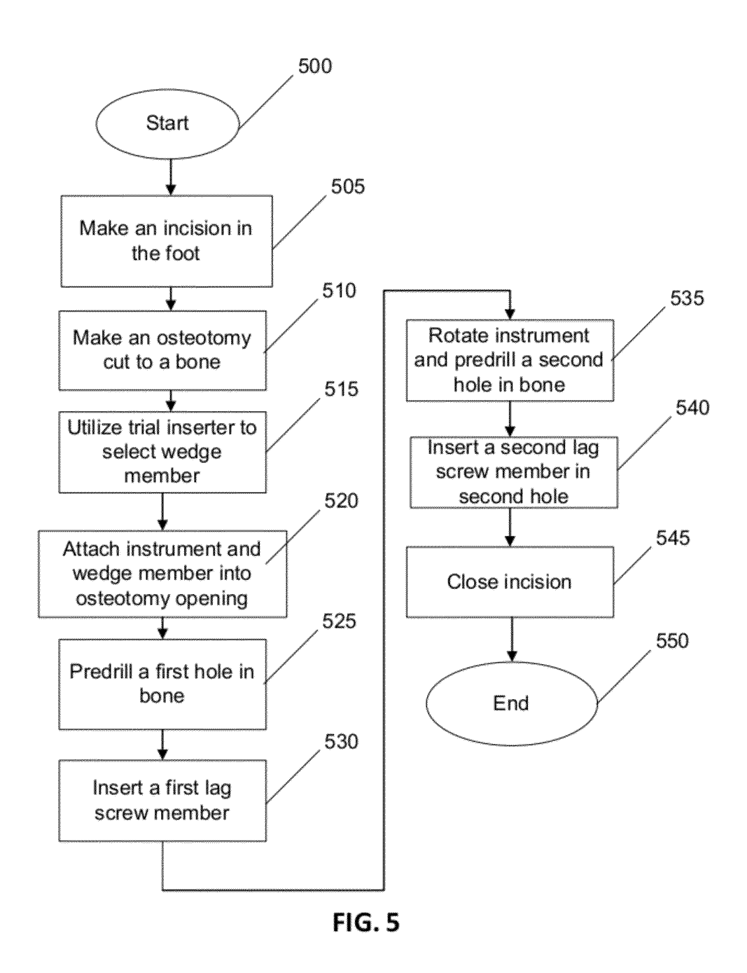

In operation, and as shown in FIGS. 4A and 5, the fixation assembly 100 may be utilized in an opening osteotomy for selectively applying an angular correction to, in one non-limiting embodiment, the metatarsal bone 105. As shown, the method starts in step 500 and proceeds to step 505, whereby a central incision is made in the lateral area of the metatarsal bone 105 of the human foot 110 in order to gain access to the metatarsal bone 105. Next, in step 510, an osteotomy cut is made in the proximal metatarsal bone 105 and a wedge opening 402 is formed by rotating the metatarsal bone 105 away from the lateral area (i.e., towards the second metatarsal bone 404). Additional instruments, such as distracters, retractors, or other similar instruments may be utilized to gain adequate exposure to the osteotomy and proper adjustment for insertion of the wedge member 205 (FIG. 2A). Next, in step 515 (as shown in FIGS. 4B and 5), a trial inserter device 406 is used to assist the surgeon in selecting the correct-sized wedge member 205 for insertion (shown in FIG. 2A) by evaluating the wedge opening 402 in the metatarsal 105. In other non-limiting embodiments, the trial inserter device 406 may differ depending on which of the metatarsal, cuneiform, calcaneous, and cuboid bones fixation is required. Next, in step 520, an instrument assembly 408 (FIG. 4C-4D) is attached to the selected wedge member 205, and the wedge member 205 is inserted into the wedge opening 402 to act as a containment for a biologic. The wedge member 205 partially fills the interstitial void in the wedge opening 402, leaving a triangular-shaped gap at the non-exposed end of the osteotomy. As shown in detail in FIG. 4D, the instrument assembly 408 has a generally elongated and cylindrical body member 410 terminating in a connecting member 412. In addition, the connecting member 412 includes a protrusion 414 adapted for actively locking onto wedge member 205 by coupling protrusion 414 to slot 345 (FIG. 3A) of wedge member 205. Also, the instrument assembly 408 includes a drill guide member 416 having a bore that is aligned with aperture 350 along axis 360 (FIG. 3A). The instrument assembly 408 may also be rotated to 180 degrees with respect to wedge member 205 in order to align guide member 416 with aperture 355 of axis 365 (FIG. 3A). The drill guide member 416 is provided to align the pre-drilled screw hole made in the underlying bone with apertures 350, 355 (FIG. 3A), thereby eliminating the mal-alignment of these screw holes with apertures 350, 355 (FIG. 3A). Next, in step 525, a hole is predrilled in the metatarsal 105 (FIGS. 4E and 4F) by inserting a pilot drill 418 through the bore in drill guide member 416 and into the corresponding aperture 350 (FIG. 3A), and forming a pilot hole in the underlying metatarsal bone 105. Next, in step 530, the screw member 210 is inserted into aperture 350 (FIGS. 3A and 4G) and into the predrilled hole, and selectively rotated to cause it to travel into the metatarsal bone 105 until rigid connection with the metatarsal bone 105 is made. Next, in step 535, the instrument assembly 408 (FIGS. 4G and 4H) is rotated 180 degrees causing the drill guide member 416 to be aligned with axis 365 of aperture 355 (FIG. 3A). A hole is now predrilled in the metatarsal 105 (FIG. 4H) utilizing the drill guide member 416. It should be appreciated that the wedge member 205 remains intact throughout the remaining steps. Next, in step 540, the screw member 215 (FIG. 2A) is inserted into aperture 355 (FIGS. 1 and 3A) and into the predrilled hole causing it to travel into the metatarsal bone 105 until rigid connection with the metatarsal bone 105 is made. Next, in step 545, the incision is closed. The method ends in step 550. It should be appreciated that the fixation assembly 100 may also be used in opening osteotomies within each of the metatarsals, cuneiform, calcaneous, cuboid, or other ankle bones.

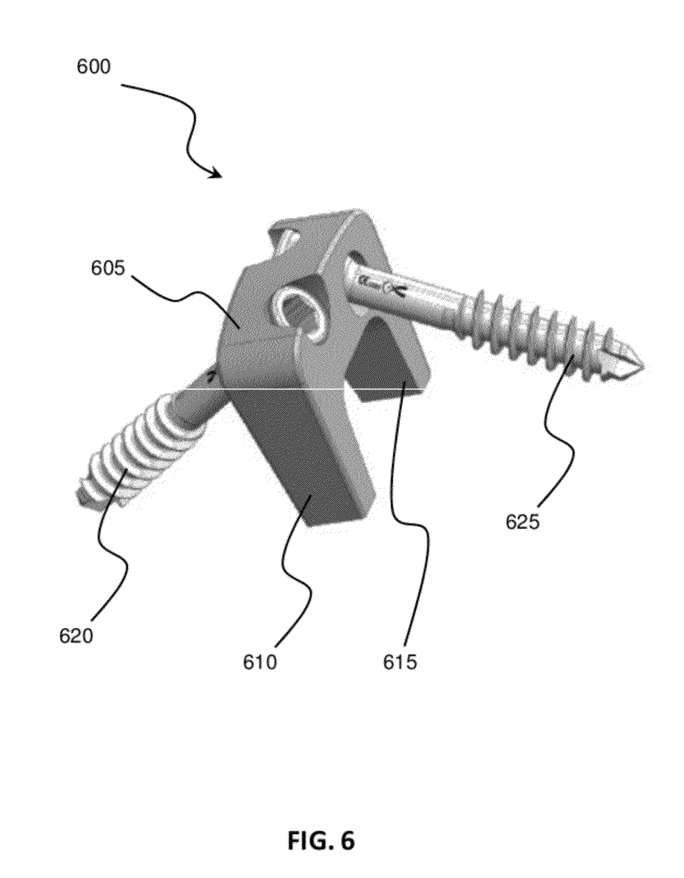

In an alternate embodiment, as shown in FIG. 6, the fixation assembly 600 may be provided having a with a “divot tool” shaped member 605, while all other aspects of the fixation assembly 600 being the same as the fixation assembly 100 shown and described in FIGS. 1-5. As shown, member 605 includes a plurality of protrusions 610 and 615 in order to couple fixation assembly 600 to other indications, for example, calcaneal side, lapidus, or metatarsal osteotomies. The member 605 may be made from a Titanium material, although, in other non-limiting embodiments, the member 605 may be made from SST, PEEK, NiTi, Cobalt chrome, or other similar types of materials. It should be appreciated that member 605 may be provided at varying lengths for the internal fixation of a variety of bone sizes in the human body. The lag screw members 620, 625 may be coated with an osteoconductive material, such as, for example, plasma spray or other similar types of porous materials that is capable of supporting or encouraging bone ingrowth into this material.

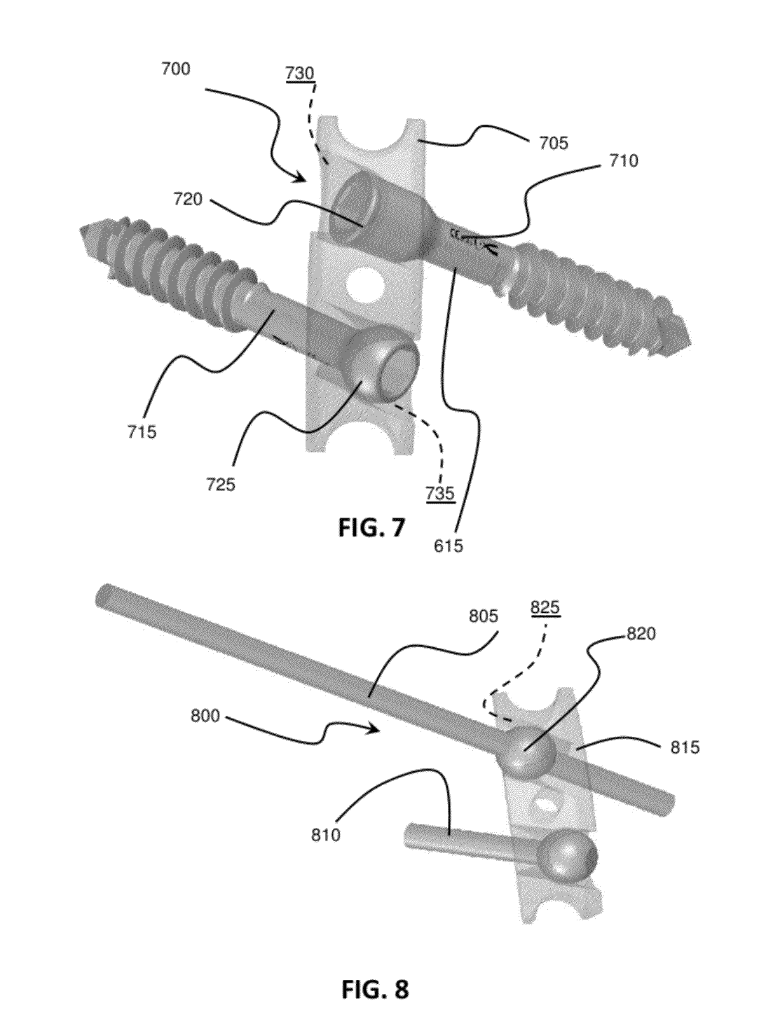

In an alternate embodiment shown in FIG. 7, the fixation assembly 700 includes a “wedge shaped” member 705 (or wedge member 705), a tapered lag screw member 710, and a polyaxial screw member 715, while all other aspects of fixation assembly 700 are substantially similar to the fixation assembly 100 that is shown and described in FIGS. 1-5. Particularly, wedge member 705 is coupled to an oblique tapered lag screw member 710 and an oblique polyaxial lag screw member 715 through corresponding apertures in the wedge member 705. The tapered lag screw member 710 includes a tapered head portion 720 with a morse taper for locking the head portion 720 to a corresponding tapered opening 730 at a fixed angle within the underlying bone. Similarly, the polyaxial lag screw member 715 includes a spherical head portion 725 for coupling the polyaxial lag screw member 715 to a spherical opening 735 at a variable angle during insertion and locking the polyaxial screw member 715 at a fixed angle after insertion into underlying bone. The lag screw members 710, 715 are inserted into apertures provided at divergent and predetermined angles and thereafter into bone in order to provide for cortical purchase of the lag screw members 710, 715 into underlying bone for fixing the fixation assembly 700 to underlying bone. The lag screw members 710, 715 may be cannulated or have a solid body and may be of a Titanium material, although, in other embodiments, the screw members 710, 715 may be provided in SST, PEEK, Ni TI, Cobalt Chrome or other similar types of materials.

In another embodiment shown in FIG. 8, the fixation assembly 800 may be utilized with a plurality of olive wires 805, 810 (also called Kirschner wires) that are each coupled to wedge member 815 at divergent angles. Particularly, the olive wire 805 includes an elongated body with a threaded portion at one end and a spherical member 820 provided in elongated body for coupling to spherical aperture 825, thereby creating an interference lock with wedge member 815. Further, olive wire 805 may be trimmed after insertion into bone, as is shown with olive wire 810. It should be appreciated that fixation assembly 800 may be made from a Titanium material, although, in other non-limiting embodiments, fixation assembly 800 may be made from SST, PEEK, NiTi, Cobalt chrome or other similar types of materials.

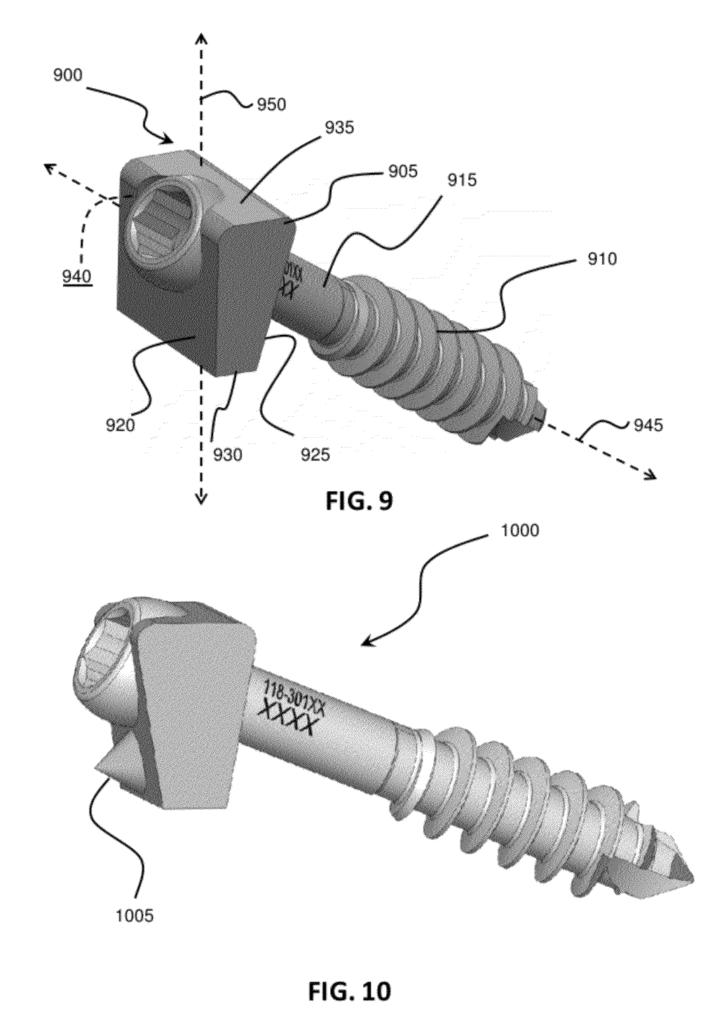

In another alternate embodiment shown in FIG. 9, the fixation assembly 900 includes a “wedge shaped” member 905 (or wedge member 905) coupled to a lag screw member 910, while all other aspects of fixation assembly 900 being substantially similar to the fixation assembly 100 that is shown and described in FIGS. 1-5. Wedge member 905 has a generally solid body portion 915 having six sides or faces. Particularly, body portion 915 extends from tapered side 920 to tapered side 925, with a width that gradually increases from side 930 to side 935, thereby giving body portion 915 a wedge shape. Also, wedge member 905 includes a through-hole or aperture 940 that is aligned along axis 945, which is at a divergent angle to the longitudinal axis 950. Similar to the other fixation assemblies, the wedge member 905 and lag screw member 910 may be made form a Titanium material, SST, PEEK, Ni TI, Cobalt Chrome, or other similar types of materials.

In another alternate embodiment shown in FIG. 10, the fixation assembly 1000 is substantially similar to the fixation assembly 900 shown and described in FIG. 9, however, includes a “conical shaped” member 1005 provided on a tapered side. The member 1005 provides a secondary point of fixation within the underlying bone.

It should be appreciated that the fixation assembly 100 maybe inserted into any of the bones of the skeletal system of foot 110 such as, but not limited to the metatarsal, cuneiform, calcaneus, and cuboid bones, in order to provide angular correction to the aforementioned bones. It should be appreciated that the fixation assembly 100 is inserted through a lateral metatarsal incision, thereby reducing the disruption to the surrounding tissues and/or the metatarsal heads while at the same time minimizing the tension on the skin. This allows for improved wound closure, reduced operating room time, reduction in the number of incisions required and reduction in the total length of incisions. It should also be understood that this invention is not limited to the disclosed features and other similar method and system may be utilized without departing from the spirit and the scope of the invention. While the invention has been described with reference to the preferred embodiment and alternative embodiments, which embodiments have been set forth in considerable detail for the purposes of making a complete disclosure of the invention, such embodiments are merely exemplary and are not intended to be limiting or represent an exhaustive enumeration of all aspects of the invention. The scope of the invention, therefore, shall be defined solely by the following claims. Further, it will be apparent to those of skill in the art that numerous changes may be made in such details without departing from the spirit and the principles of the invention. It should be appreciated that the invention is capable of being embodied in other forms without departing from its essential characteristics.

1. An intraosseous fixation assembly, comprising:
a wedge member for creating a resultant angle in an osteotomy, wherein said wedge member includes a body portion and a plurality of apertures;
a first lag member adapted for coupling to said body portion at a first angle; and
a second lag member adapted for coupling to said body portion at a second angle;
wherein said wedge member is generally trapezoid in shape and partially fills an interstitial space in said osteotomy; and
wherein said wedge member includes a top surface for disposition in an anterior portion of said osteotomy and a bottom surface that is opposed to said top surface for disposition in a posterior portion of said osteotomy.

2. The intraosseous assembly of claim 1, wherein each of said first and said second angle is in a range of about 0 degrees to about 90 degrees with said body portion.

3. The intraosseous assembly of claim 1, wherein said plurality of apertures includes at least a first aperture and a second aperture.

4. The intraosseous assembly of claim 3, wherein said first aperture is adapted for receiving said first screw member.

5. The intraosseous assembly of claim 3, wherein said second aperture is adapted for receiving said second screw member.

6. The intraosseous assembly of claim 1, wherein said body portion includes a first radius of curvature at said top surface and a second radius of curvature at said bottom surface.

7. The intraosseous assembly of claim 6, wherein said first radius of curvature is greater than said second radius of curvature.

8. The intraosseous assembly of claim 1, wherein said body portion includes a first perimeter at said top surface and a second perimeter at said bottom surface, wherein said first perimeter decreases from said first surface to said second perimeter at said bottom surface.

9. The intraosseous assembly of claim 1, wherein said body portion comprises a bore aligned along a bore axis, wherein said bore axis is orthogonal to a plane that is parallel to a plane traversing longitudinally through said body portion.

10. The intraosseous assembly of claim 9, wherein said bore is provided to receive a complementary shaped end of an instrument.

11. The intraosseous assembly of claim 1, wherein each of said first and second lag members is a Kirschner wire.

12. The intraosseous assembly of claim 1 wherein each of said first and said second lag members is a screw member.

13. The intraosseous assembly of claim 11, wherein each of said first and second members includes a bulbous head portion for providing an interference fit with said apertures.

14. The intraosseous assembly of claim 13, wherein said bulbous head portion includes a morse taper for providing said interference fit.

15. The intraosseous assembly of claim 13, wherein said bulbous head portion includes an orifice longitudinally coextensive with a length of said bulbous head portion.

16. The intraosseous assembly of claim 15, wherein said orifice has a hexagonal shape, a star shape, or a square shape.

17. The intraosseous assembly of claim 15, wherein said orifice is provided to receive a complementary shaped end of a screw instrument.

18. The intraosseous assembly of claim 1, wherein each of said first and second lag members includes a threaded portion having a plurality of bone threads on an outer surface thereof.

19. The intraosseous assembly of claim 11, wherein each of said first and second lag members includes a spherical portion for providing an interference fit with said apertures.

20. A method for angular correction of a bone, comprising the steps of:
providing a wedge member having a first aperture and a second aperture;
forming an osteotomy in a first metatarsal bone;
spreading the osteotomy to create a cavity;
coupling a targeting guide assembly to the wedge member;
inserting the wedge member into the cavity at a predetermined depth;
inserting a drill member into the targeting guide assembly and into the first aperture;
forming a first hole in the metatarsal bone;
inserting a first lag screw member into the first aperture and into the first hole and compressing the metatarsal bone;
inserting the drill member into the targeting guide assembly and into the second aperture;
forming a second hole in the metatarsal bone; and
inserting a second lag screw member into the second aperture and into the second hole and compressing the metatarsal bone.

21. The method of claim 20, wherein the targeting guide assembly is removed prior to inserting of each of the lag screw members.

22. The method of claim 20, further comprising forming the osteotomy in a lateral cortex of the first metatarsal bone.

23. The method of claim 20, where the wedge member comprises a body portion and a plurality of apertures.

24. The method of claim 20, further comprising inserting the first lag screw member at a first angle, the first angle is in a range of about 0 degrees to about 90 degrees with the wedge member.

25. The method of claim 20, further comprising inserting the second lag screw member at a second angle, the second angle is in a range of about 0 degrees to about 90 degrees with the wedge member.

26. The method of claim 20, wherein the wedge member includes a first radius of curvature at a top surface of the wedge member and a second radius of curvature at an opposed bottom surface of the wedge member.

27. The method of claim 26, wherein the first radius of curvature is greater than the second radius of curvature.

28. The method of claim 26, wherein the wedge member includes a first perimeter at the top surface and a second perimeter at the bottom surface, the first perimeter decreasing from the top surface to the second perimeter at the bottom surface.

29. The method of claim 20, wherein each of the lag screw members includes a bulbous head for providing an interference fit with each of the apertures.

30. The method of claim 29, wherein the bulbous head includes a morse taper for providing the interference fit.

31. The method of claim 20, wherein each of the lag screw members includes a threaded portion having a plurality of bone threads on an outer surface thereof.

32. An intraosseous fixation assembly, comprising:
a wedge member for creating a resultant angle in an osteotomy, wherein said wedge member includes a body portion having a plurality of sides and a bore aligned along a bore axis; and
a lag screw member adapted for coupling to said body portion within said bore.

33. The intraosseous assembly of claim 32, wherein the body portion includes a first side having a first perimeter and an opposed second side having a second perimeter, said first perimeter being greater than said second perimeter.

34. The intraosseous assembly of claim 33, wherein the first side is adapted for positioning at an anterior end of said osteotomy and the second side is adapted for positioning at a posterior end of said osteotomy.

35. The intraosseous assembly of claim 32, wherein said bore includes an opening at a common edge connecting said first side and an adjacent side.

36. The intraosseous assembly of claim 35, wherein said opening is tapered for receiving a tapered lag screw.

37. The intraosseous assembly of claim 35, wherein said opening is tapered for receiving a polyaxial lag screw member.

38. The intraosseous assembly of claim 35, wherein said bore emanates from a third side that is directly opposite said adjacent side.

39. The intraosseous assembly of claim 32, wherein said body portion includes a conical member that is provided on said adjacent side.

40. The intraosseous assembly of claim 32, wherein said bore axis is at an acute angle with a longitudinal axis of said body portion.

41. The intraosseous assembly of claim 40, wherein said acute angle is in a range of about 0 degrees to about 90 degrees.

42. The intraosseous assembly of claim 32, wherein said body portion includes a tapered opening

43. The intraosseous assembly of claim 32, wherein said screw member includes a bulbous head for providing an interference fit with said bore.

44. The intraosseous assembly of claim 43, wherein said bulbous head includes a morse taper for providing said interference fit.

45. The intraosseous assembly of claim 43, wherein said bulbous head includes a spherical portion for providing said interference fit.

46. The intraosseous assembly of claim 44, wherein said bulbous head includes an orifice longitudinally coextensive with a length of said bulbous head.

47. The intraosseous assembly of claim 46, wherein said orifice has a hexagonal shape, a star shape, or a square shape.

48. The intraosseous assembly of claim 46, wherein said orifice is provided to receive a complementary shaped end of an instrument.

49. The intraosseous assembly of claim 32, wherein said lag screw member includes a threaded portion with a plurality of bone threads on an outer surface thereof.

50. The intraosseous assembly of claim 32, wherein said wedge member is provided for insertion into an osteotomy of a bone.
